(19) United States
(12) Patent Application Publication
(10) Pub. No.: US 2026/0218011 A1
(43) Pub. Date: Jul. 30, 2026

(54) MUSHROOM MYCELIUM MAT-BASED ELASTOMER ANALOG AND METHOD FOR PREPARING SAME

(71) Applicant: INDUSTRY-ACADEMIC COOPERATION FOUNDATION, CHOSUN UNIVERSITY, Gwangju (KR)

(72) Inventors: Hyun Jae SHIN, Gwangju (KR); Da Song KIM, Gwangju (KR); Yong Hyeon JEONG, Gwangju (KR)

(21) Appl. No.: 19/144,051

(22) PCT Filed: Oct. 31, 2023
(86) PCT No.: PCT/KR2023/017089

§ 371 Date: Jun. 27, 2025

(30) Foreign Application Priority Data
Dec. 28, 2022 (KR) 10-2022-0187651

(51) Int. Cl. C09D 189/00

(52) U.S. Cl. CPC C09D 189/00

(57) ABSTRACT

The present invention relates to a method for preparing an elastomer analog using a mushroom mycelium mat and an elastomer analog by using a mushroom mycelium mat, and to an elastomer analog on the basis of a mushroom mycelium mat prepared by the method. The method for preparing an elastomer analog on the basis of a mushroom mycelium mat of the present invention may effectively prepare an elastomer analog having Young's modulus and density properties that correspond to those of an elastomer material through a stepwise and sequential process. In addition, a mushroom mycelium mat-based elastomer analog prepared by the method may be effectively used as an eco-friendly material that can replace a synthetic elastomer.

### TECHNICAL FIELD

The present disclosure relates to a method for preparing an elastomer analogue using a mushroom mycelium mat and a mushroom mycelium mat-based elastomer analogue prepared by the method.

### BACKGROUND ART

Recently, as awareness of environmental protection has increased worldwide, there is a movement to replace plastic products. Plastic is a word derived from the Greek word ‘plastikos’, meaning ‘easily processed into a desired shape’, and the definition of the word is ‘a polymer compound that may be molded by applying heat and pressure’. In addition, plastic is a type of polymer, and polymers are classified into thermoplastic resin, thermosetting resin, and elastomer according to characteristics. Among these, elastomer is a high value-added polymer material that has the properties of rubber and plastic and may be easily processed, characterized by excellent elasticity, resilience, shock absorption, etc. Elastomers may be classified into thermosetting and thermoplastic depending on some characteristics.

In addition, elastomers are a representative material used in functional films, building soundproofing materials, cable coverings, shoe absorbent layers, packaging materials, cushioning materials, rubber, etc., and are widely used in daily life. Various types of polymers such as plastics and elastomers are difficult to biodegrade, causing significant environmental pollution upon disposal. Waste polymers are generally treated by incineration, landfill, recycling, etc., but numerous hazardous materials and greenhouse gases are emitted during incineration processes, landfilling causes environmental pollution, and recycling rates are not high.

Therefore, the introduction of eco-friendly biodegradable materials is urgent, and as research on biomass-based biodegradable materials is actively progressing, the use of mushroom mycelia is receiving considerable attention. Even currently, mushroom mycelia have been used in various fields such as packaging materials, building materials, alternative leather, alternative meat, etc., and are showing sufficient functionality as substitutes. Regarding mushroom mycelia, research on containers or cushioning materials that replace Styrofoam, alternative leather, etc. has progressed considerably, but research on elastic materials such as elastomers is lacking.

Since existing elastomers require advanced technology for production itself, elastomers can only be produced only by specific companies. In contrast, mushroom culture does not require advanced technology, and the most important aspect is to establish a culture system that may eradicate contamination and maintain an optimal growth environment. In addition, since mycelial film has the characteristic of forming a dense mesh structure with hyphae and becoming tough, mycelial film is targeted as a mushroom mycelium-based biodegradable substitute that has elastomeric properties by adding appropriate physical and chemical treatment processes. In a previous study related to this, a biofilm exhibiting elastomeric properties was disclosed, prepared by drying Schizophyllum commune mycelium and then immersing the mycelium in glycerol at a concentration of 16% or higher. However, if only a single glycerol solution is used, the performance sustainability of a mycelium-based material may be reduced. In fact, the present inventors confirmed that the surface was partially contaminated with fungi, etc. after a mycelial film of Trametes orientalis mycelium was immersed in a glycerol solution and then left for a long time indoors (drawing not disclosed). Therefore, an additional treatment process is needed to reduce these disadvantages.

In the present disclosure, a mushroom mycelium mat-based elastomer analogue was intended to be developed as an eco-friendly material capable of replacing existing synthetic elastomers, and as a result, an elastomer analogue having physical properties corresponding to elastomer material in terms of Young's modulus and density was prepared through a stepwise and sequential process using a mat in which aerial hyphae and biofilm hyphae among parts of the edible mushroom mycelium grew to exhibit high density, thus completing the present disclosure.

### DISCLOSURE

### Technical Problem

Therefore, the present disclosure is directed to providing a method for effectively preparing a mushroom mycelium mat-based elastomer analogue as an eco-friendly material capable of replacing a synthetic elastomer.

The present disclosure is further directed to providing a mushroom mycelium mat-based elastomer analogue as an eco-friendly material capable of replacing a synthetic elastomer prepared by the method.

### Technical Solution

An aspect of the present disclosure provides a method for preparing a mushroom mycelium mat-based elastomer analogue, the method including preparing a mushroom mycelium mat, pretreating the mushroom mycelium mat with an alkali aqueous solution, surface-modifying the mushroom mycelium mat subjected to the pretreatment process, and treating the surface-modified mushroom mycelium mat with a plasticizer and a coating agent and then heat-pressing the treated mushroom mycelium mat.

In one embodiment of the present disclosure, the mushroom mycelium may be a Trametes orientalis mycelium.

In one embodiment of the present disclosure, the alkali aqueous solution may be a sodium hydroxide aqueous solution.

In one embodiment of the present disclosure, the surface-modifying may include modifying the mushroom mycelium mat subjected to the pretreatment process with one surface modification solution selected from the group consisting of acetic anhydride, benzoyl chloride, and 3-aminopropyltriethoxysilane.

In one embodiment of the present disclosure, the plasticizer may be polyethylene glycol.

In one embodiment of the present disclosure, the coating agent may be corn-zein.

In one embodiment of the present disclosure, the heat-pressing may be performed at 100 to 120° C. for 5 to 15 minutes.

Another aspect of the present disclosure provides a mushroom mycelium mat-based elastomer analogue prepared by the method.

In one embodiment of the present disclosure, the elastomer analogue may have a Young's modulus of 2 to 2.5 N/mm² and a density of 1 to 1.5 g/cm³.

### Advantageous Effects

According to the present disclosure, a method for preparing a mushroom mycelium mat-based elastomer analogue can effectively prepare an elastomer analogue with Young's modulus and density properties corresponding to the physical properties of an elastomer material through stepwise and sequential processes. In addition, the mushroom mycelium mat-based elastomer analogue prepared by the method can be usefully used as an eco-friendly material capable of replacing a synthetic elastomer.

### DESCRIPTION OF DRAWINGS

FIG. 1 is a schematic diagram showing a structure of a mycelium consisting of aerial hyphae, biofilm hyphae, and penetrative hyphae.

FIG. 2 is a photograph of a liquid-cultured mushroom mycelium (Trametes orientalis (Yasuda) Imazeki) transferred to a plastic box and cultured for 28 days in a dark room under conditions of 28° C. and 80-90% humidity.

FIG. 3 is a photograph of a mushroom mycelium mat collected after a substrate culture of a mushroom mycelium (Trametes orientalis (Yasuda) Imazeki) is completed.

FIG. 4 is a schematic diagram showing a process for preparing a mushroom mycelium mat-based elastomer analogue of the present disclosure.

FIG. 5A is a photograph of a mushroom mycelium mat pretreated with an alkali solution (non-treated: non-treated group, Alkali treated: alkali pretreated group).

FIG. 5B is a photograph of an alkali-pretreated mushroom mycelium mat chemically surface-modified with various surface modification solutions (Siane treated: 3-aminopropylthreethoxysilane treated group, Benzoylation: benzoyl chloride treated group, Acetylation: acetic anhydride treated group).

FIG. 5C is a photograph of a mushroom mycelium mat pretreated with an alkali solution, surface-modified with various surface modification solutions, coated with a plasticizer and then heat-pressed (Siane treated: 3-aminopropylthreethoxysilane treated group, Benzoylation: benzoyl chloride treated group, Acetylation: acetic anhydride treated group).

FIG. 6 is a SEM image showing surface shapes of a non-treated group (NT) and mushroom mycelium mat-based elastomer analogue samples (ACHP, BCHP, and SCHP) of the present disclosure.

FIG. 7 shows results of measuring water contact angles of a non-treated group (NT) and mushroom mycelium mat-based elastomer analogue samples (ACHP, BCHP, and SCHP) of the present disclosure.

FIG. 8 shows results of thermogravimetric analysis of a non-treated group (NT) and mushroom mycelium mat-based elastomer analogue samples (ACHP, BCHP, and SCHP) of the present disclosure.

FIG. 9 is a graph showing material properties of a mushroom mycelium mat-based elastomer analogue of the present disclosure in the material classification chart (Material properties chart, Source: CES EduPack 2019, ANSYS Granta © 2020 Granta Design) based on Young's modulus-density.

### BEST MODE

The present disclosure provides a method for preparing a mushroom mycelium mat-based elastomer analogue.

In the present disclosure, “mushroom mycelium” refers to an organ through which mushrooms absorb nutrients for survival. Mushrooms consist of mycelia, which are vegetative organs, and fruiting bodies, which contain spores as reproductive organs, and the mycelia correspond to roots/stems/leaves in general plants, while fruiting bodies correspond to flowers.

The cell wall of the mushroom mycelium has a structure in which polysaccharides (α-1,3-glucan) and glycoproteins (mainly mannan or galactomannoproteins) are connected to an amorphous gel-like matrix on a membrane woven with microfibers of chitin and β-1,3 glucan. Chitin and β-1,3-glucan are key components that support the structure of the cell wall, with chitin playing a particularly crucial role in the mechanical strength of hyphal fibers.

During growth, mushroom hyphae have nutrient affinity, and grow by ingesting nutrients while expanding tips. After the hyphae expand, the tips are formed on the cell wall to cause branches, and the branches extend in a continuous and characteristic radial pattern. These branches extend in various directions to cause numerous fusions between hyphae. The fusion of hyphae is uniform in terms of material properties, and in terms of mechanical properties, the mechanical stiffness increases with the formation of a hyphal micro-network. The mushroom mycelium is formed by interwoven hyphae that create a network layer with a microstructure similar to the dermis layer of the animal skin. However, the hyphal density is relatively low, which results in limited durability and strength.

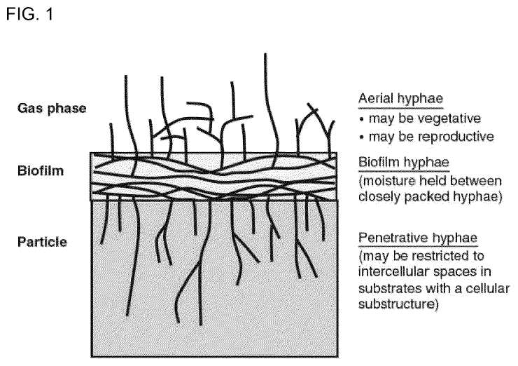

The hyphae may be broadly classified into three types: of aerial hyphae, biofilm hyphae, and penetrative hyphae (see FIG. 1). The aerial hyphae play a role in exchanging oxygen and carbon dioxide required for mycelial growth, and the biofilm hyphae are located between the aerial hyphae and the penetrative hyphae and have a dense mat shape. The biofilm hyphae prevent moisture loss from the medium, prevent the invasion of other fungi, and store moisture for mycelial growth. As the thickness and density of the biofilm increase, nutrient transfer (mass transfer) in the medium part becomes difficult, and the growth of the biofilm and the aerial hyphae stops. Meanwhile, the penetrative hyphae serve to secrete enzyme substances to decompose nutrients in the medium and ingest the decomposed nutrients.

In the present disclosure, the term “mushroom mycelium” refers narrowly to biofilm hyphae and more broadly to a concept that includes both aerial hyphae and biofilm hyphae, among the three types: aerial hyphae, biofilm hyphae, and penetrative hyphae.

In the present disclosure, a mat form in which the aerial hyphae and the biofilm hyphae of the mushroom mycelium grow to create a high-density mat is referred to as the “mushroom mycelium mat.”

In the present disclosure, the “mushroom mycelium mat-based elastomer analogue” refers to an analogue having elastomeric physical properties prepared using the mushroom mycelium mat.

A method for preparing a mushroom mycelium mat-based elastomer analogue of the present disclosure may include (a) preparing a mushroom mycelium mat, (b) pretreating the mushroom mycelium mat with an alkali aqueous solution, (c) surface-modifying the mushroom mycelium mat subjected to the pretreatment process, and (d) treating the surface-modified mushroom mycelium mat with a plasticizer and a coating agent and then heat-pressing the treated mushroom mycelium mat.

The Step (a) of the present disclosure is a step of preparing a mushroom mycelium mat, which can be prepared either by direct production or by purchasing a pre-made one.

For example, the mushroom mycelium mat may be prepared in the form of a high-density mat by inoculating and then culturing the mushroom mycelium into a medium to grow aerial hyphae and biofilm hyphae.

In one embodiment of the present disclosure, the mushroom mycelium may be a mushroom mycelium of one type selected from the group consisting of Laetiporus sulphureus, Microporus affinis, Ganoderma lucidum, Schizophyllum commune, Trametes versicolor, Daedaleopsis tricolor, Elfvingia applanata, Mycoleptodonoides aitchisonii, Fomitopsis pinicola, Agaricus arvensis, Lentinula edodes, Wolfiporia extensa, and Trametes orientalis, preferably a Trametes orientalis mycelium.

The Step (b) of the present disclosure is a step of pretreating the mushroom mycelium mat, and more specifically, a step of pretreating the mushroom mycelium mat with an alkali aqueous solution to weaken the structure of the mushroom mycelium mat and increase the permeability of the treatment solution.

In one embodiment of the present disclosure, the alkali aqueous solution may be a sodium hydroxide (NaOH) aqueous solution.

The Step (c) of the present disclosure is a step of modifying the surface of the mushroom mycelium mat, and more specifically, a step of modifying the mushroom mycelium mat subjected to the pretreatment process through Step (b) with a surface modification solution to impart various functional groups to the mushroom mycelium mat and change the physical properties. An unprocessed mushroom mycelium mat has only hydrophilic properties, and in order to implement the physical properties of an elastomer corresponding to an elastic plastic material and to be applied to various high value-added materials, it is required to control surface hydrophilic properties.

In one embodiment of the present disclosure, the surface modification solution may be one solution selected from the group consisting of acetic anhydrous, benzoyl chloride, and 3-aminopropyltriethoxysilane.

The Step (d) of the present disclosure is a step of treating the mushroom mycelium mat with a plasticizer and a coating agent and then heat-pressing the treated mushroom mycelium mat. Specifically, the surface-treated mushroom mycelium mat subjected to Step (c) may be treated with a plasticizer and a coating agent to impart elasticity and flexibility to the mushroom mycelium mat, increase tensile strength, and increase the adhesion with the coating agent through heat pressing.

In one embodiment of the present disclosure, the plasticizer may be polyethylene glycol (PEG), and the coating agent may be a corn zein-based solution, but the type thereof is not particularly limited.

In another embodiment of the present disclosure, the heat-pressing may be performed at 100 to 120° C. for 5 to 15 minutes.

Further, the present disclosure provides a mushroom mycelium mat-based elastomer analogue prepared by the method.

The mushroom mycelium mat-based elastomer analogue may have a Young's modulus of 2 to 2.5 N/mm² and a density of 1 to 1.5 g/cm³.

The mushroom mycelium mat-based elastomer analogue may have hydrophobic interfacial properties.

The mushroom mycelium mat-based elastomer analogue may have various interfacial properties depending on a type of surface modification.

Specifically, in the case of the mushroom mycelium mat-based elastomer analogue (BCHP) surface-modified with benzoyl chloride, since the contact angles of the top and bottom are almost similar values, it is suitable to be used as a material having uniform interfacial properties of the inside and outside. In addition, in the case of the mushroom mycelium mat-based elastomer analogue (SCHP) surface-modified with 3-aminopropylthreethoxysilane, the top shows a tendency for a hydrophilic property to further decrease than the bottom, and thus it was determined that it may be utilized as a material with different interfacial properties, where the outside has a low affinity for water and the inside has a high affinity for water.

Therefore, the mushroom mycelium mat-based elastomer analogue of the present disclosure, prepared depending on a type of surface modification, may be used according to an application purpose.

In one embodiment of the present disclosure, the mushroom mycelium mat-based elastomer analogue may also be used as interior products such as cushioning packaging materials or indoor mattress.

Hereinafter, the present disclosure will be described in more detail with reference to Examples. However, these Examples are more provided to more specifically illustrate the present disclosure, and the scope of the present disclosure is not limited to these Examples.

[Modes]

### Example 1

### Preparation and Culture of Mushroom Strain

### <1-1> Preparation of Mushroom Strain

In this experiment, Trametes orientalis (Yasuda) Imazeki obtained from the Jeollanam-do Forest Resources Research Institute was used and stored on a yeast malt agar (YMA) medium.

### <1-2> Suitable Mycelial Culture

A culture substrate was mixed with 80% oak sawdust and 20% rice bran, the moisture content was adjusted to about 55%, and then the mixture was placed in a plastic box (155×155×87 mm, HPL822D, LOCK & LOCK), sterilized at 121° C. for 60 minutes, and then used. At this time, several holes with a diameter of 1 cm were drilled into the cover of the plastic box and attached with filters. The liquid-cultured Trametes orientalis (Yasuda) Imazeki mycelium was ground with a blender, centrifuged at 3000 rpm for 20 minutes to remove the supernatant, and then added with a YMB medium as a fresh nutrient source. The mycelial solution was inoculated onto the substrate and cultured in a dark room of 27° C. under 80-90% humidity conditions for 4 weeks to produce a mushroom mycelium mat. The produced mushroom mycelium mat was collected and dried.

For reference, the “mushroom mycelium mat” refers to a mat form in which the aerial hyphae and biofilm hyphae of the mushroom mycelium grow to form a dense mat.

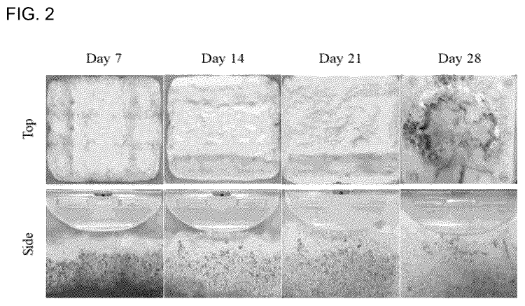

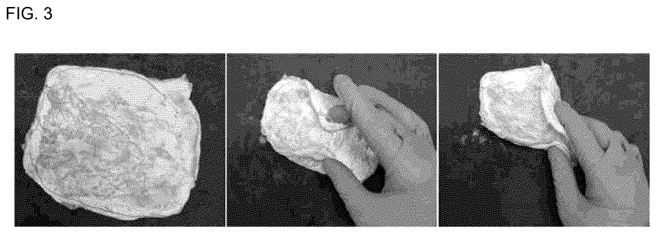

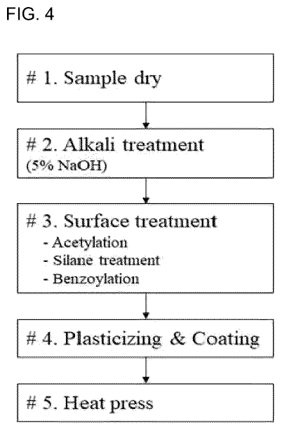

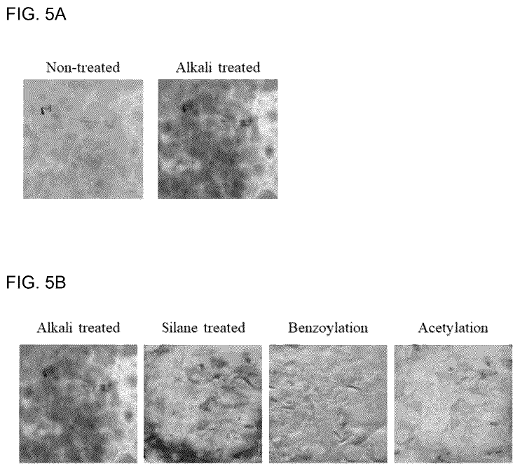

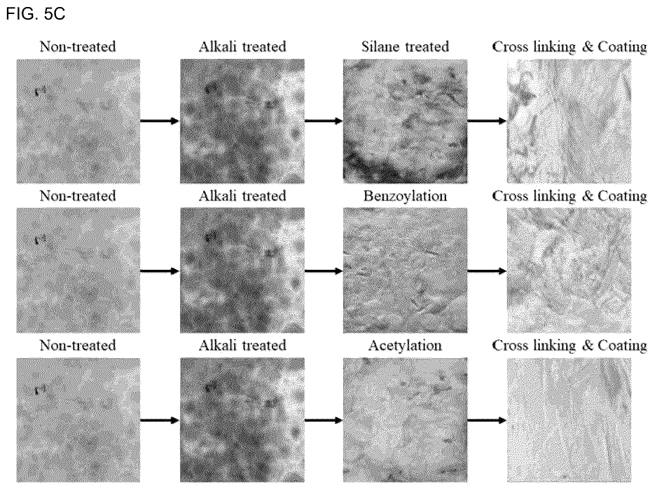

FIG. 2 is a photograph of a liquid-cultured mushroom mycelium (Trametes orientalis (Yasuda) Imazeki) transferred to a plastic box and then cultured in a dark room under conditions of 28° C. and 80-90% humidity for 28 days, and FIG. 3 is a photograph of a mycelial film collected after a substrate culture of the mushroom mycelium (Trametes orientalis (Yasuda) Imazeki) was completed.

### Example 2

### Preparation of Mushroom Mycelium Mat-Based Elastomer Analogue

### <2-1> Alkali Pretreatment

In order to weaken the structure of the mushroom mycelium mat prepared through the culture of <Example 1> above and increase the permeability of a treatment solution, the mushroom mycelium mat was immersed in a 5% (w/v) NaOH aqueous solution for 15 minutes. The alkali treatment was completed, the mycelial film was washed with distilled water before use. The alkali treatment was intended to weaken the interwoven structure of the mycelium and facilitate the effective action of the subsequent treatment solution.

### <2-2> Surface Modification Treatment

The mushroom mycelium mat treated with alkali through Example <2-1> above was immersed in a surface modification solution for a certain period of time. The surface modification solution was used with 2% (v/v) 3-aminopropyltriethoxysilane (Daejung, Korea), acetic anhydride (Duksan, Korea), and 10% (v/v) benzoyl chloride (Daejung, Korea), respectively. At this time, benzoyl chloride was prepared using pure ethanol (100% ethanol) as a solvent, and 3-aminopropyltriethoxysilane was prepared using 60% ethanol as a solvent at corresponding concentrations, respectively, and used. Acetic anhydride was used as a stock solution. The samples were immersed for 30 minutes, and when benzoyl chloride was used, the samples were additionally immersed in 70% ethanol for 30 minutes. The mushroom mycelium mats that had been immersed in the solution were washed several times with distilled water and dried at 60° C. for 1 hour to prepare a total of three types of surface-modified mushroom mycelium mats.

### <2-3> Plasticizing, Coating, and Heat-Pressing

PEG 400 (Sigma-Aldrich Korea) was dissolved in 70% ethanol to prepare a 20% (v/v) polyethylene glycol solution, and then corn-zein (Sigma Aldrich) was added to the polyethylene glycol solution to prepare a plasticizing & coating solution with a final concentration of 15% (w/v). The three types of surface-modified mushroom mycelium mats prepared through Example <2-2> were treated with the plasticizing & coating solution for 3 hours. Thereafter, the mushroom mycelium mats were washed with distilled water and dried at 60° C. for 1 hour. Finally, the mushroom mycelium mats were subjected to a heat pressing process at 100 to 120° C. and 500 to 1000 psi for 10 minutes by using a heated press (Carver 4386 CH Laboratory Manual Heated press, Carver Inc., USA) to obtain the mushroom mycelium mat-based elastomer analogue of the present disclosure.

TABLE 1
Mushroom mycelium mat-based elastomer
analogue of the present disclosure

| Sample abbreviation | Surface modification solution type |
|---|---|
| ACHP | Acetic anhydride |
| BCHP | Benzoyl chloride |
| SCHP | 3-Aminopropyltriethoxysilane |

### Experimental Example 1

### Measurement of Physical Properties of Mushroom Mycelium Mat-Based Elastomer Analogue

The physical properties (tensile strength, elongation, density, and Young's modulus) of the mushroom mycelium mat-based elastomer analogue of the present disclosure prepared through <Example 2> above were measured. The tensile strength, elongation, and Young's modulus were measured using a UTM (AGS-X, Shimadzu, Japan) at a tensile speed of 100±20 mm/min after 80×20 mm mycelium mat samples were prepared. In addition, the density was measured using a gas pycnometer (Ultrapyc 5000 Foam, Anton Paar, Austria) at 3.0 psi and 20° C.

TABLE 2
Physical properties of mushroom mycelium mat-based elastomer analogue

| | Tensile strength | Elongation | Density | Young's modulus |
|---|---|---|---|---|
| Sample | (N/mm²) | (%) | (g/cm³) | (N/mm²) |
| NT | 1.134 ± 0.238 | 14.764 ± 1.339 | 1.178 ± 0.014 | 1.040 ± 0.157 |
| ACHP | 2.593 ± 0.133 | 31.020 ± 1.495 | 1.315 ± 0.016 | 2.323 ± 0.125 |
| BCHP | 2.762 ± 0.090 | 29.686 ± 2.446 | 1.384 ± 0.011 | 2.443 ± 0.079 |
| SCHP | 2.305 ± 0.135 | 27.799 ± 1.697 | 1.347 ± 0.017 | 2.062 ± 0.128 |

NT: Non-treated group
ACHP: Mushroom mycelium mat-based elastomer analogue surface-modified with acetic anhydride
BCHP: Mushroom mycelium mat-based elastomer analogue surface-modified with benzoyl chloride
SCHP: Mushroom mycelium mat-based elastomer analogue surface-modified with 3-aminopropyltriethoxysilane

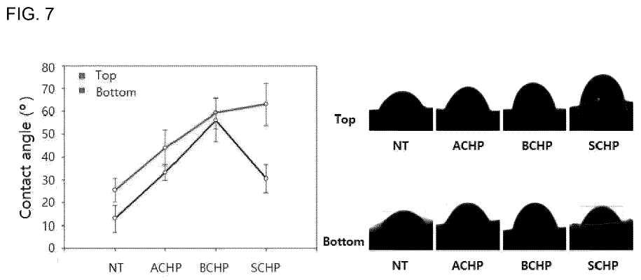

The mushroom mycelium mat harvested initially in the experiment had a very strong hydrophilic property, whereas it was confirmed that the hydrophilic property gradually decreased through the alkali treatment (Example <2-1>), surface modification treatment (Example <2-2>), plasticizing and coating treatment & heat-pressing process (Example <2-3>) (see FIG. 7). Specifically, in the case of the mushroom mycelium mat-based elastomer analogue (BCHP) surface-modified with benzoyl chloride, it was determined that since the contact angles of the top and bottom were almost similar values, it was suitable to be used as a material having uniform interfacial properties of the inside and outside. In addition, in the case of the mushroom mycelium mat-based elastomer analogue (SCHP) surface-modified with 3-aminopropylthreethoxysilane, the top showed a strong hydrophobic property and the bottom showed a relatively close hydrophilic property, and thus it was determined that it was suitable to be utilized as a material with different interfacial properties, where the outside had a low affinity for water and the inside had a high affinity for water.

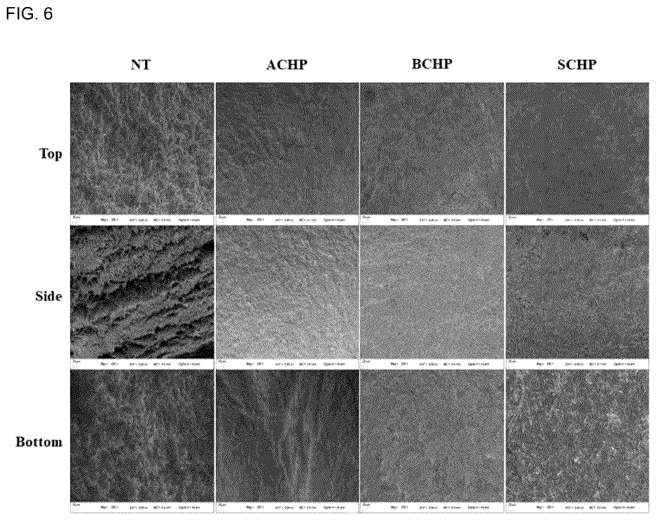

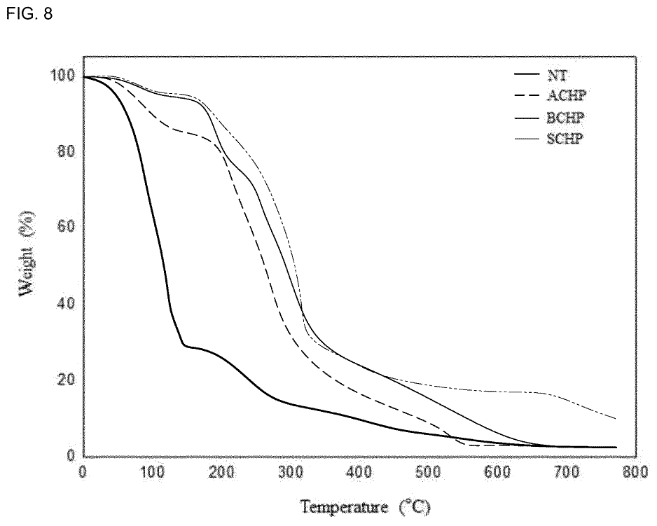

In addition, the surface of the mushroom mycelium mat-based elastomer analogue had a significantly uniform shape (see FIG. 6), and a weight change at a high temperature was noticeably reduced compared to the non-treated sample (see FIG. 8).

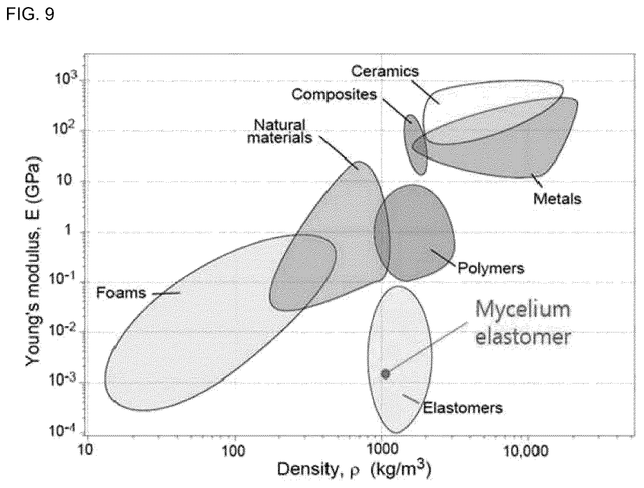

Meanwhile, the physical property classification of the mushroom mycelium mat-based elastomer analogue was shown in FIG. 9. As shown in FIG. 9, the mushroom mycelium mat-based elastomer analogue of the present disclosure exhibited the physical properties of the elastomer material.

Hereinabove, the present disclosure has been described with reference to preferred embodiments thereof. It will be understood by those skilled in the art that the present disclosure may be implemented as modified forms without departing from an essential characteristic of the present disclosure. Therefore, the disclosed embodiments should be considered from an illustrative viewpoint rather than a restrictive viewpoint. The scope of the present disclosure is illustrated by the appended claims rather than by the foregoing description, and all differences within the scope of equivalents thereof should be construed as being included in the present disclosure.

### [National Research and Development Project Supporting the Invention]

[Project Unique Number] 1405005402
[Project Number] 2020191B10-2222-BA01
[Ministry name] Korea Forest Service
[Project Management (Special) Institution Name] Korea Forestry Promotion Institute
[Research Project Name] Research on discovery of forest biomaterials
[Research Subject Name] Design of forest mushroom mycelium incubator and development of mycelium treatment technology
[Percent Contribution] 1/1
[Project performance institute name] Chosun University's Industry-Academic Cooperation Foundation
[Research Period] 2022.01.01 to 2022.12.31.

1. A method for preparing a mushroom mycelium mat-based elastomer analogue, the method comprising:
preparing a mushroom mycelium mat;
pretreating the mushroom mycelium mat with an alkali aqueous solution;
surface-modifying the mushroom mycelium mat subjected to the pretreatment process; and
treating the surface-modified mushroom mycelium mat with a plasticizer and a coating agent and then heat-pressing the treated mushroom mycelium mat.

2. The method of claim 1, wherein the mushroom mycelium is a Trametes orientalis mycelium.

3. The method of claim 1, wherein the alkali aqueous solution is a sodium hydroxide aqueous solution.

4. The method of claim 1, wherein the surface-modifying comprises modifying the mushroom mycelium mat subjected to the pretreatment process with one surface modification solution selected from the group consisting of acetic anhydride, benzoyl chloride, and 3-aminopropyltriethoxysilane.

5. The method of claim 1, wherein the plasticizer is polyethylene glycol.

6. The method of claim 1, wherein the coating agent is corn-zein.

7. The method of claim 1, wherein the heat-pressing is performed at 100 to 120° C. for 5 to 15 minutes.

8. A mushroom mycelium mat-based elastomer analogue prepared by the method of claim 1.

9. The mushroom mycelium mat-based elastomer analogue of claim 8, wherein the elastomer analogue has a Young's modulus of 2 to 2.5 N/mm² and a density of 1 to 1.5 g/cm³.

10. The method of claim 1, wherein the elastomer analogue exhibits hydrophobic surface properties with a water contact angle greater than 90°.

11. The method of claim 1, wherein the surface-modified mycelium mat shows interfacial asymmetry between top and bottom surfaces based on contact angle measurement.

12. The mushroom mycelium mat-based elastomer analogue of claim 8, wherein the material is used in cushioning packaging or indoor mattress applications.
